SEQUENCE LISTING (28 sequences)

SEQ ID NO: 1 - DNA, 21 bp, Artificial Sequence
  /note=“SEQ ID NO. 1 (tumour specific promoter element motif), wherein r = g or a, y = t or c, m = a or c, k = g or t, w = a or t, s = g or c, b = t or g or c, v = a or g or c, h = a or t or c, d = a or t or
yhnydtkttwttwanasrwam

SEQ ID NO: 2 - DNA, 21 bp, Artificial Sequence
  /note=”SEQ ID NO. 2 (tumour specific promoter element motif)“
cagtattttatttaaaggtaa

SEQ ID NO: 3 - DNA, 21 bp, Artificial Sequence
  /note=”SEQ ID NO. 3 (tumour specific promoter element motif), wherein r = a or g“
ccatattttatttaraggtaa

SEQ ID NO: 4 - DNA, 21 bp, Artificial Sequence
  /note=”SEQ ID NO. 4 (tumour specific promoter element motif)“
ccatattttatttagaggtaa

SEQ ID NO: 5 - DNA, 472 bp, Artificial Sequence
  /note=”SEQ ID NO. 5 (4ydiH)“
gatctttgatgaacagggacgtctgtgctgttcttcgcgtcttactacagcgattgtgtgacgttgtcaccataaaagtgtgatgcatattgcttttggtcaaaatgtgcgcagaaaaaggtgatatactgtgcacgtttacacataagcgaggatgatatgtctactgatttggacccaacccaactggcgattgaatttttacgccgtgataaaaccgaactttctcccgcgcagtatttgaagcgtttaaaacagctggagttagagtttgccgatctcctcaccctctcagcaaccgaactgaaagaagagatctatttcgcctggcggttgggcgtgcattaataaagtctgtgtaggccggataaggcgttagccgccatccggctatgttaccaggcaatctcaccagaccttgtccccaggtaatagataacccccttgattttgtttaaaaaaaatacagatc

SEQ ID NO: 6 - DNA, 21 bp, Artificial Sequence
  /note=”SEQ ID NO. 6 (tumor-specific promotor element of 4ydiH)“
ttttgtttaaaaaaaatacag

SEQ ID NO: 7 - DNA, 1152 bp, Artificial Sequence
  /note=”SEQ ID NO. 7 (t48gIpT)“
gatcgatggcttcagcgccaatgcctgccgcttcacaggcgcgaataaaggtggcctgaaacgcaagatcatcttccggcaacgtaataaacagaccatccgtcggttcgacgcaatggcgggcgatccgttttaaaatctggttttcactaatgcactcgcgcgcggattccgcgtcggtaaccgcataacgagcgccgctgtgcagcagcccgtggttacgcccggtcgcgccggtcgctatatcatgccgctccaccagaatgacacgtaaaccgcgcagcgcgcagtcgcgggcgatccctgcgcctgttgctccaccgccaatgataatcacgtcacttgtttgcgagtcgcgagttttcattgtttttcctcacagttcgttttttatcatttagccatacaaatcatatgtaatgtttgatttcgcgcataatcgctcactattcgaaaatgaaacgtgatttcgtgcgcctttctgaacattagtcataaatctgtaacaatatgtgctgtaattcacattaacgtgacgtatctttacttaacatcgcggccacactgggagcagcggggttgtttgaacgaactgcggtgtttacctctaaataaaatatgggccacggaggctacaatatgttgagtatttttaaaccagcgccgcataaagcgcgcttgccagcggcggagattgatccgacctatcgccgattacgctggcagattttcctggggatattctttggctatgccgcgtattatctggtgcgcaagaactttgccctcgcgatgccctacctggtagaacagggtttttcacgcggcgatctgggctttgcgctgtccgggatttccatcgcttatggtttttcgaaatttataatgggttcggtgtccgatcgctcgaatccgcgcgtttttctgccggcagggttgattctggccgcagcagtcatgttgtttatgggctttgtgccgtgggcgacatccagcatcgccgtgatgtttgtactgttgttcctttgcggctggttccaggggatggggtggccgccgtgcggtcgtacgatggttcactggtggtcgcagaaagagcgcggcggcattgtgtcggtctggaacggcgcgcataacgtcggcggcgggatc

SEQ ID NO: 8 - DNA, 21 bp, Artificial Sequence
  /note=”SEQ ID NO. 8 (tumor-specific promotor element of 48GIpT)“
ttacctctaaataaaatatgg

SEQ ID NO: 9 - DNA, 1093 bp, Artificial Sequence
  /note=”SEQ ID NO. 9 (92yhjU)“
gatcgctttacaggcgaacccccttctcaatcgtcatcggatgatttacggtacgttaacggcggaactttccacaaccgtacatgcgctggcactgcatacttataccctcaaagagtgggaaggattgcaggatactatcttcgcgtcgcctccttgtcgcggcgcgggaagcatcgcgtagcaaaacggatttgcaactacctccgcttttccagtatgttgctacagaattatgtgaaaacggcctgcgggccgttttgttttgtctgaattttgagcgtgtcgtacagtattcagacaaaaattagccgagaattgtgaaaaccgccgcagcatcgcacaatcaccgttctcgactcacaaaagtgatgccgctataatgcgccgtcttatatatgaacgtcttcgggatgattctgacgacagggaatgtgattgattacgagaacatcccggttccgcgaagcaaatagcacgtgcttgcggagtagagttgaccgagcactgtgattttttgaggtaacaagatgcaagtttcagttgaaaccactcaggggccttggccgccgtgtaacgattacaatcgctgctgacagcatcgagaccgctgtaaaaagcgagctggtcaacgtagcgaaaaaagtacgtattgacggcttccgtaaaggcaaagtaccgatgaatatcgtcgctcagcgttatggcgcttctgttcgccaggacgtgctgggcgatcattccctggaacgccccgctttcacgctgcctgacgcttattgaaagcgtgcaggggcagcagttcagccgttacgtaccggaagacatcaccacgctactgtcgatgacgcagccgttgaaactgcgcggttttcagccgtgggataccttctgcgatgccatccatacgatgatgagcaacaccctgctccccgccgacgggaaaggcgttctggtcgcgctgcgcccggtgccgggcattcgggttgagcaggcgttaacattatgtcggccaaacagggccggcgatattatgaccatcggcggcaaccgtctggaaaggtggttatcattctgccggggtctaacgatc

SEQ ID NO: 10 - DNA, 21 bp, Artificial Sequence
  /note=”SEQ ID NO. 10 (tumor-specific promotor element of 92yhjU)“
cactgtgattttttgaggtaa

SEQ ID NO: 11 - DNA, 715 bp, Artificial Sequence
  /note=”SEQ ID NO. 11 (135dniR)“
gatcccggcgaagcggcccccgtcttaaaggccattgctgaacataagtggatgccggaggcgatttttttgacgcatcaccatcacgaccatgttggcggagtcaaagagctgttgcaacacttcccgcaaatgacggtttatggaccggcggaaacgcaagacaagggagcaacccatcttgttggcgatggcgatactattcgcgttttaggcgagaaatttactctttttgccacgccgggccacacgttaggacacgtctgttactttagccgcccttacttattctgcggcgacacgctgttttccggcggctgtggtcgactgtttgaaggcacgccatcacagatgtatcagtcacttatgaaaattaactctctgcctgacgacacgctcatttgctgcgctcacgaatacactttagctaacattaagttcgcattgagcatacttccgcacgattcgttcataaatgaatattatcgtaaagttaaagagttacgtgtaaaaaaacaaatgacattacccgttattcttaaaaatgagcgtaagattaatctttttttaagaactgaagatattgatttaattaacgaaataaacaaagaaacaatattgcaacaaccagaagcgcgttttgcatggttaaggtcaaagaaagacacgttctgataattcttacttgtcattcgctaacttcgccgttatgatc

SEQ ID NO: 12 - DNA, 21 bp, Artificial Sequence
  /note=”SEQ ID NO. 12 (tumor-specific promotor element of 134dniR)“
ttacgtgtaaaaaaacaaatg

SEQ ID NO: 13 - DNA, 466 bp, Artificial Sequence
  /note=”SEQ ID NO. 13 (154mdh)“
gatccggattacgcaaattaaatgcataaaagccaaaattgcgcgactccgcattcttgatgagtgaggattgtaatcattgaatttgtgaattaaggtcgccgccgcggagcaatagacacttagctaatcatataataaggagtttaggatgaaagtcgcagtcctcggcgctgctggtggtatcggtcaggcgctggcattacttttaaaaaaccaactgccttcaggttcagaactctccctgtacgacatcgctccagtgactcccggtgtggccgttgatttgagccacatccccaccgctgtaaaaatcaaaggtttctccggtgaagacgcaaccccggcgcttgaaggcgctgacgtagtactgatttctgcgggtgtggcgcgtaagccgggtatggaccgttccgacctgtttaacgttaacgccggcatcgtgaaaaacctggtgcagcagatc

SEQ ID NO: 14 - DNA, 21 bp, Artificial Sequence
  /note=”SEQ ID NO. 14 (tumor-specific promotor element of 154mdh)“
ttacttttaaaaaaccaactg

SEQ ID NO: 15 - DNA, 564 bp, Artificial Sequence
  /note=”SEQ ID NO. 15 (156yeel)“
gatcgcaaggtgaaaatgagcccaaccctggacaggaagcgttgagcttttcgatgtgcgccagttaaaattctggcgtttttttctcaccgaattttctcattttttctcaacgtgattttcatcactataagaaaatcacgtaagtgcttgaatagtggcggagagagagggattcgaaccctcggcggagttacccccgcaacggttttcgagaccggtccgttcagccgctccggcatctctccgtatattgcaatgatgccaggtaatttggcattttaacagaccctattcgggtaattttgttcaagtgacgagtttacgagcaaaacgatgattaagtggccctggaaagcacaagaaataacccagaacgaagactggccgtgggatgatgcgctggctatacctcttctggtaaacctcaccgcgcaagaacaggctcggcttattgcgctagccgaacgttttttgcagcagaaaagactggtagcgctacagggatttgagctcgactcgttaaaaagtgcacgtattgcgttaattttttgcttaccgatc

SEQ ID NO: 16 - DNA, 21 bp, Artificial Sequence
  /note=”SEQ ID NO. 16 (tumor-specific promotor element of 156yeel)“
tctcattttttctcaacgtga

SEQ ID NO: 17 - DNA, 441 bp, Artificial Sequence
  /note=”SEQ ID NO. 17 (172frdA)“
gatcggcaaagaaacgacggatttccgccataatcgccgcacgttttaataaattggggatggacgcgctcggctgccaggttgccgtttcgctcatgattctttctccagtttaagacaaggtcacgaagtctactcgcaacgcgcgggcgaaacaaattttgcgcaggcgtatcgggcgccttctggagggtaaaaaaagtgatttcagatggtttagtaattaaattaatcaaaatcaatgataattcatccctctgatacgctaaaaaaatcgaacacgtcaaatttccctcacatccctgagactatactgttgtacccataaaggagcagtggaaacgcattcatacttcgcagaacccagaggctttatctggctgcgcgagggtgaaattacaataatctggaggaatgtcgtgcaaacctttcaagccgatc

SEQ ID NO: 18 - DNA, 21 bp, Artificial Sequence
  /note=”SEQ ID NO. 18 (tumor-specific promotor element of 172frdA)“
gcacgttttaataaattgggg

SEQ ID NO: 19 - DNA, 614 bp, Artificial Sequence
  /note=”SEQ ID NO. 19 (185pfkA)“
gatcgcgcgctgcccgatgcggaacgacaggaaattattgatatcgtgacgtcatggccgggagtcagcggcgcgcacgatctccgcacgcggcagtcagggccgactcgctttattcagattcatttggaaatggaagataatctgccgctcgttcaggcgcattttgtggctgaccagattcatttggaaatggaagataatctgccgctcgttcaggcgcattttgtggctgaccagtgttcagtcgttcccagggaaggcaggaagttcgagcttgtataattgattgttaaaaagtgagccaggccagcattttgtgtataaattaccgccatttggcctgacctgaatcaattcagcaggaagggattgttatactatctgtatattcgttggatcgtttcgaagtgcgaaatcggcttccggcaatagatttcattttgcattccaaagttcagaggtagtcatgattaagaaaatcggtgtgttgacaagcggcggtgatgcgccgggcatgaacgcggcaatccgcggtgttgtgcgcgcagcgttgacggaagggctggaagtcatgggcatttatgacggctatctgggcctgtatgaagatc

SEQ ID NO: 20 - DNA, 21 bp, Artificial Sequence
  /note=”SEQ ID NO. 20 (tumor-specific promotor element of 185pfkA)“
cagcattttgtgtataaatta

SEQ ID NO: 21 - DNA, 437 bp, Artificial Sequence
  /note=”SEQ ID NO. 21 (212pflE)“
gatcggttcgctacaggcaatggaggccattaagctactggcgcattacggtcagcctgccagcggaaaaatcgtcatgtacgatgcgatgacctgtcagttccgcgaaatgaagttaatgcgcaaccccggctgtgaggtctgcgggcagtagccactttacggataaacataccaaagcacggtttatggccgtgctgtatcaacagataattactgtcatcagacgcaaagccagcttacccaagctccgactgacgatcggctcaagcagcgcggcatcatcttaaacgccataccacaacctcaaaccgtgatgttgtacctcatttaacgcctctttgtcagaacctctccattcgttgacgcacatcaagatagctttcattcgaaagtaatttaatctttatatgaaataagagaggccgtttatgatc

SEQ ID NO: 22 - DNA, 20 bp, Artificial Sequence
  /note=”SEQ ID NO. 22 (tumor-specific promotor element of 212pflE)“
taatctttatatgaaataag

SEQ ID NO: 23 - DNA, 556 bp, Artificial Sequence
  /note=”SEQ ID NO. 23 (nirB)“
gatcgcagcaaacgaaagtggcctcgcctaagctggttaacttcattctgacctgcggcaccatcggaacaatgctgaccttcgtcgtcaccggcccgattgtagcgcacagcggcccacaggcggcgttactcaccgcgaatggtctgtatgcggtggtctttgtgatgtgctttgcgctcggctttgtatcccgtcatcgtcagcatagcgcgccggctacgcattgataatccttgccggatggagacatcgccctccggcactctatcccccctcctgacggggtaggcctgttggtctaaaaacccctcattttgtatgttatttgtacaaacctgaaaagcctgacaattccgccacttataaaaatccagacaaatcagccatatacccattaagaggtatataaaggtgaatttgatttacatcaataagcggggttgctgaatcgttaaggtaggcggtaatagaaaagaaatcgaggcaaaaatgagcaaagtcagactcgctattatcggtaatggtatggtcggccaccgctttattgaggatc

SEQ ID NO: 24 - DNA, 21 bp, Artificial Sequence
  /note=”SEQ ID NO. 24 (tumor-specific promotor element of 271nirB)“
ttgtatgttatttgtacaaac

SEQ ID NO: 25 - DNA, 708 bp, Artificial Sequence
  /note=”SEQ ID NO. 25 (301glpA)“
gatcgccgcgtgaaaaaccctgttctaccaggtagggcatcgcgagggcaaagttcttgcgcaccagataatacgcggcatagccaaagaatatccccaggaaaatctgccagcgtaatcggcgataggtcggatcaatctccgccgctggcaagcgcgctttatgcggcgctggtttaaaaatactcaacatattgtagcctccgtggcccatattttatttagaggtaaacaccgcagttcgttcaaacaaccccgctgctcccagtgtggccgcgatgttaagtaaagatacgtcacgttaatgtgaattacagcacatattgttacagatttatgactaatgttcagaaaggcgcacgaaatcacgtttcattttcgaatagtgagcgattatgcgcgaaatcaaacattacatatgatttgtatggctaaatgataaaaaacgaactgtgaggaaaaacaatgaaaactcgcgactcgcaaacaagtgacgtgattatcattggcggtggagcaacaggcgcagggatcgcccgcgactgcgcgctgcgcggtttacgtgtcattctggtggagcggcatgatatagcgaccggcgcgaccgggcgtaaccacgggctgctgcacagcggcgctcgttatgcggttaccgacgcggaatccgcgcgcgagtgcattagtgaaaaccagattttaaaacggatc

SEQ ID NO: 26 - DNA, 21 bp, Artificial Sequence
  /note=”SEQ ID NO. 26 (tumor-specific promotor element of 301glpA)“
ccatattttatttagaggtaa

SEQ ID NO: 27 - DNA, 581 bp, Artificial Sequence
  /note=”SEQ ID NO. 27 (310ybaL)“
gatcgtatctttttataaaacacaaccattttttataaacatcctgattgaaattgtcataaactattacccggagttttggagtccagcaaccaaaggagacggaatgcatcacgctaccccgcttatcaccaccattgtcggcggccttgtgctcgcttttattctcggcatgattgccaacaaattgcgtatttctccactggtgggatatctgttagcgggcgttctggcgggaccttttaccccgggttttgttgcggataccaaactggcgccggagctggcggagctcggcgtgattctattgatgttcggcgtcgggctgcatttttcgctgaaggatttgatggcggtaaagtctatcgccattcccggcgctgtcgctcagatagcggtggcgacgctgctgggtatggcgctttccgccgtgctgggatggtcattaatgaccggcatcgtttttgggctctgtctgtctacagccagtaccgtcgtcctgctgcgcgcgcttgaggagcgacaactccttgatagccagcgcgggcaaatcgccatcggctggctgattgtcgaagatc

SEQ ID NO: 28 - DNA, 21 bp, Artificial Sequence
  /note=”SEQ ID NO. 28 (tumor-specific promotor element of 310ybaL)“
gtatctttttataaaacacaa

(19) United States
(12) Patent Application Publication
(10) Pub. No.: US 2012/0244621 A1
(43) Pub. Date: Sep. 27, 2012

(54) TUMOR-SPECIFIC BACTERIAL PROMOTER ELEMENTS

(75) Inventors: Siegfried Weiss, Braunschweig (DE); Sara Bartels, Braunschweig (DE); Holger Loessner, Langen (DE); Igor Deyneko, Braunschweig (DE); Dirk Bumann, Riehen (CH); Kathrin Westphal-Daniel, Braunschweig (DE)

(73) Assignee: HELMHOLTZ-ZENTRUM FUR INFEKTIONSFORSCHUNG GmbH, Braunschweig (DE)

(21) Appl. No.: 13/321,777

(22) PCT Filed: May 28, 2010
(86) PCT No.: PCT/EP2010/057452

§ 371 Date: Feb. 6, 2012

(30) Foreign Application Priority Data
May 28, 2009 (EP) 09161442.0

(51) Int. Cl. C12N 1/21; C12N 15/70; C12N 15/74

(52) U.S. Cl. 435/471; 435/252.3; 435/252.33; 435/252.31; 435/252.2; 435/252.32

(57) ABSTRACT

The invention relates to bacterial promoter elements which constitute or which are comprised in promoter regions and which confer tumour specificity to the promoter region activity, resulting in transcription of a transgene, which can be a heterologous or a homologous gene, which transgene is functionally arranged downstream of the promoter region at presence of a host bacterium in tumour tissue, while essentially conferring inactivity of the promoter region and no transcription at presence of the host bacterium in non-tumour tissue. Accordingly, the invention relates to bacterial promoter regions containing these tumour specific promoter elements.

The present invention relates to bacterial promoter elements which constitute or which are comprised in promoter regions and which confer tumour specificity to the promoter region activity, resulting in transcription of a transgene, which can be a heterologous or a homologous gene, e.g. a human gene or a Salmonella gene, which transgene is functionally arranged downstream of the promoter region at presence of a host bacterium in tumour tissue, while essentially conferring inactivity of the promoter region and no transcription at presence of the host bacterium in non-tumour tissue. Accordingly, the invention relates to bacterial promoter regions containing these tumour specific promoter elements. The tumour specific activity of the promoter elements is that their presence in a promoter region makes the promoter region specifically inducible by the localization of a host bacterial vector in tumour tissue. A bacterial promoter of the invention further confers a significantly reduced activity to a promoter region containing it, preferably no activity at presence of the bacterial vector in non-tumour tissue, i.e. the promoter regions containing a promoter element of the invention are significantly less or essentially not induced at presence of the host bacterial vector in non-tumour tissue. For the purposes of this invention, tumours preferably are solid tissue tumours and/or metastases, e.g. benign and malignant tumours, e.g. solid cancer including metastatic tumour tissue. For use in the treatment of cancer, the invention therefore relates to bacterial vectors containing an expression cassette, wherein a nucleic acid sequence that can be transcribed is arranged under the control of a promoter region comprising or consisting of a tumour specific promoter element. Further, the invention relates to a bacterial vector containing an expression cassette encoding a transgene and a promoter controlling the transgene, which promoter contains the tumour specific promoter element, for use as medicament, e.g. for tumour treatment, and to the use of the tumour specific promoter elements in the production of a pharmaceutical composition containing a bacterial vector for use in the treatment of a solid tumour. Preferably, the tumour is a solid tumour.

### STATE OF THE ART

Arrach et al. in Cancer Research 68, 4827-4832 (2008) describe a method for screening Salmonella enterica typhimurium genomic DNA fragments of 300 to 500 bp using a reporter gene encoding GFP to fragments that lead to expression of the reporter in tumour samples of a tumour bearing experimental animal, but not in spleen. As a result, single nucleotides are identified, and the flanking genes are mentioned. Without giving nucleotide sequences, Arrach et al. describe that three of the promoter sections showed enhanced reporter gene expression when growing in tumour relative to spleen in an in vivo test. A multitude of putative tumour-specific nucleotide positions and their flanking genes is given in supplementary table 2 of Arrach et al, without indication of a defined region that could have effect as a regulatory element.

The general use of bacterial vectors as a pharmaceutical agent, wherein the promoter region is selected according to its induction pattern is described in WO 99/55364, wherein the arabinose inducible promoter P_{BAD} is used for control of transgene expression, in Bowers et al. (Gene, 11-18, 2004) using the P_{BAD} promoter for control of plasmid copy number in a bacterial vector, and in Wong et al. (Gene 117-186, 2003) using a xylose inducible promoter for tight regulation of the expression of an essential gene in Haemophilus influenzae.

Löβner et al. (Expert Opinion on Biological Therapy, 157-168, 2004) describe DNA transfer for gene therapy and vaccination by bacterial vectors. Dietrich et al. (Current Drugs, 10-19, 2003) describe live attenuated bacterial vaccines. WO 2006/079790 describes gut bacteria that comprise a promoter which is induced by presence of a dietary factor to produce a biologically active protein. WO 99/55364 describes the production of antigenic MxiM protein of Shigella for use as a vaccine, inter alia by expression under the control of inducible promoters P_{BAD} and P_{LAC}. WO 2006/048344 describes a bacterial vector containing an expression cassette for a transgene under the control of a saccharide inducible promoter for use in tumour therapy, in which the promoter is induced by administration of the inductor saccharide concurrent to or separate from the administration of the bacterial vector.

King et al. in Human Gene Therapy 1225-1233 (2002) describe an attenuated Salmonella vector containing a gene encoding a thioredoxin-cytosine desaminase fusion protein, cloned into a commercial vector designated pTrc99A. The bacterial vector was administered to tumour-bearing mice and expressed the pro-drug converting fusion protein at sites of accumulation, i.e. in tumour tissue.

### OBJECTS OF THE INVENTION

It is an object of the invention to provide nucleotide sequences conferring tumour specificity to a promoter region, and preferably ensuring essentially no induction of promoter activity in non-tumour tissue, e.g. in spleen. A further object of the invention is to provide bacterial vectors containing the tumour specific promoter elements in an expression cassette to control the tumour specific induction of an orf arranged within the expression cassette.

### GENERAL DESCRIPTION OF THE INVENTION

The invention achieves the above-mentioned objects by providing nucleic acid sequences which confer tumour specific promoter activity with little or no promoter activity in non-tumour tissue, e.g. in spleen, when the promoter sequences are contained in or constitute the promoter region of an expression cassette contained in a bacterial vector. For example, the tumour specific promoter elements of the invention can constitute or be comprised in a promoter region of an expression cassette to control transcription of an orf, e.g. a transgene under the control of the promoter region. The tumour specific promoter elements have a modulating effect when present in a promoter region to confer activity to the promoter region at presence of the bacterium comprising the nucleotide sequence within tumour tissue, and preferably conferring inactivity to a promoter region when the bacterium is present in non-tumour tissue like spleen.

Further, the invention provides bacterial vectors containing an expression cassette having at least one of the promoter elements of the invention to control the transcription of an orf, e.g. a transcribable nucleotide sequence like a transgene, under the control of a promoter region comprising or consisting of a promoter element of the invention, which bacterial vector is suitable for use as a pharmaceutical composition or a medicament, e.g. in the treatment of tumours.

Accordingly, the invention also provides the use of the promoter elements in the production of a pharmaceutical composition, and a process for production of a pharmaceutical composition or medicament, the composition or medicament containing a bacterial vector, which vector contains an expression cassette, wherein an orf, e.g. a transgene, is functionally arranged under the control of a promoter region containing or consisting of a promoter element according to the invention. Further, the invention provides pharmaceutical compositions comprising the bacterial vector of the invention for use in the medical treatment of a tumour, as well as a process for tumour treatment of a mammal, especially a human, bearing a tumour, the process comprising the administration of the bacterial vector to the mammal.

The bacterial vectors comprising an expression cassette, in which the promoter region consists of or comprises a tumour specific promoter element, are preferably part of a pharmaceutical composition for use in tumour therapy. In addition to the tumour specific promoter element, a promoter region of an expression cassette preferably comprises or consists of the −10 and the −35 regions. In general, nucleotide sequences are given in 5′ to 3′.

From a number of pre-selected DNA sections, the promoter elements of the invention have been deduced, which are characterized by their tumour specificity of inducing transcription of a transgene functionally linked downstream to a promoter region containing a tumour specific promoter element, and by the relative inactivity of a promoter region containing the tumour specific promoter element at presence of the bacterial vector in a non-tumour tissue, e.g. in spleen, for induction of transcription of a transgene under their control Therefore, the individual tumour specific promoter elements contained in each of the DNA sections having promoter activity are presently presumed to confer the tumour specificity of promoter activity of the DNA sections.

As the tumour specific element or motif of the invention has been derived from more than one natural sequence, the nucleotides within this motif have a different importance for the tumour specificity. This different importance of nucleotides is expressed by the different weights of the nucleotides as represented by the following Table 1 for SEQ ID No. 1.

TABLE 1
Position weight matrix for motif of SEQ ID No. 1

| Position | A | C | G | T |
|---|---|---|---|---|
| 1 | 0.00 | 461.86 | 0.00 | 173.20 |
| 2 | 85.48 | 85.48 | 0.00 | 64.11 |
| 3 | 14.14 | 14.14 | 35.36 | 14.14 |
| 4 | 0.00 | 131.60 | 0.00 | 592.18 |
| 5 | 241.75 | 0.00 | 69.07 | 69.07 |
| 6 | 45.21 | 0.00 | 90.42 | 361.68 |
| 7 | 0.00 | 0.00 | 173.20 | 461.86 |
| 8 | 78.03 | 0.00 | 0.00 | 780.25 |
| 9 | 0.00 | 78.03 | 0.00 | 780.25 |
| 10 | 203.35 | 0.00 | 33.89 | 135.56 |
| 11 | 0.00 | 0.00 | 0.00 | 1100.00 |
| 12 | 90.42 | 0.00 | 45.21 | 361.68 |
| 13 | 131.60 | 0.00 | 0.00 | 592.18 |
| 14 | 510.36 | 0.00 | 56.71 | 56.71 |
| 15 | 52.52 | 10.50 | 31.51 | 21.01 |
| 16 | 1100.00 | 0.00 | 0.00 | 0.00 |
| 17 | 50.51 | 101.02 | 126.27 | 0.00 |
| 18 | 251.49 | 0.00 | 301.79 | 0.00 |
| 19 | 131.60 | 0.00 | 0.00 | 592.18 |
| 20 | 361.68 | 0.00 | 45.21 | 90.42 |
| 21 | 461.86 | 173.20 | 0.00 | 0.00 |

The tumour specific promoter motif is also represented by its reverse complementary sequence, for which Table 2 gives the position weight matrix:

TABLE 2
Position weight matrix for the reverse complement of
motif of SEQ ID No. 1

| Position | A | C | G | T |
|---|---|---|---|---|
| 1 | 0.00 | 0.00 | 173.20 | 461.86 |
| 2 | 90.42 | 45.21 | 0.00 | 361.68 |
| 3 | 592.18 | 0.00 | 0.00 | 131.60 |
| 4 | 0.00 | 301.79 | 0.00 | 251.49 |
| 5 | 0.00 | 126.27 | 101.02 | 50.51 |
| 6 | 0.00 | 0.00 | 0.00 | 1100.00 |
| 7 | 21.01 | 31.51 | 10.50 | 52.52 |
| 8 | 56.71 | 56.71 | 0.00 | 510.36 |
| 9 | 592.18 | 0.00 | 0.00 | 131.60 |
| 10 | 361.68 | 45.21 | 0.00 | 90.42 |
| 11 | 1100.00 | 0.00 | 0.00 | 0.00 |
| 12 | 135.56 | 33.89 | 0.00 | 203.35 |
| 13 | 780.25 | 0.00 | 78.03 | 0.00 |
| 14 | 780.25 | 0.00 | 0.00 | 78.03 |
| 15 | 461.86 | 173.20 | 0.00 | 0.00 |
| 16 | 361.68 | 90.42 | 0.00 | 45.21 |
| 17 | 69.07 | 69.07 | 0.00 | 241.75 |
| 18 | 592.18 | 0.00 | 131.60 | 0.00 |
| 19 | 14.14 | 35.36 | 14.14 | 14.14 |
| 20 | 64.11 | 0.00 | 85.48 | 85.48 |
| 21 | 173.20 | 0.00 | 461.86 | 0.00 |

The sequence in Tables 1 and 2, which each represent the motif, is reported as the tumour specific promoter element, wherein the weight of the motif is defined by the sum of the individual nucleotide in each position of one of the above given matrices (sum of matrix values which corresponds to each nucleotide and its position), and wherein sequences of the invention, i.e. tumour specific sequences have a total weight of at least 7460, i.e. the sum of individual weights of the nucleotides of each position for the sequence is at least or higher than 7460. Accordingly, the tumour specific promoter element is defined by each of the matrices of Table 1 or Table 2 with the proviso that the sum of the matrix values for each nucleotide is at least 7460. This definition of the promoter element reflects the individual importance of nucleotides in their specific position, wherein the sum of the individual matrix values for the nucleotides in each position is at least 7460 for the tumour specific promoter elements of the invention.

The tumour specific promoter elements of the invention preferably correspond to or are represented by YHNYDTKTTWTTWANASRWAM (SEQ ID No. 1), wherein non-standard nucleotides are represented as follows: R is G or A, Y is T or C, M is A or C, K is G or T, W is A or T, S is G or C, B is T or G or C, V is A or G or C, H is A or T or C, D is A or T or G, and N is A or T or G or C, and to the reverse complementary sequence of SEQ ID No. 1.

The motif of the promoter element corresponding to SEQ ID No. 1 is closest contained in the best matching motif of SEQ ID No. 2: CAGTATTTTATTTAAAGGTAA

From the nucleic acid sections identified to contain a tumour specific promoter element of the invention, CCATATTTTATTTAGAGGTAA (SEQ ID No. 3), with R=A or G, could be inferred from SEQ ID No. 2. In one clone, the following tumour specific promoter element was identified: CCATATTTTATTTAGAGGTAA (SEQ ID No. 4). From the analysis of DNA sections with tumour specific promoter activity, SEQ ID No. 4 was found to be the closest one to the sequences corresponding to SEQ ID Nos. 1 or 2.

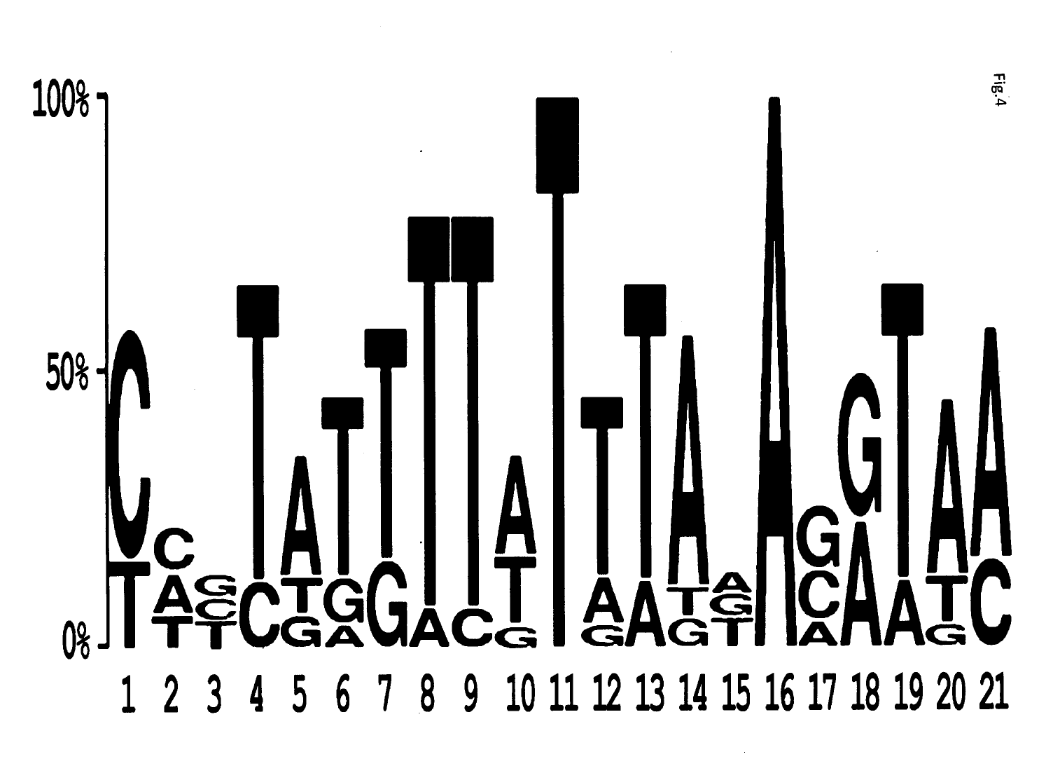

The sequence motif of the tumour specific promoter element is represented in FIG. 3 with the Y-axis indicating the incidence of the respective base at the position indicated on the X-axis.

In FIG. 4, the sequence motif for the tumour specific promoter element is shown with the relative presence of natural nucleotides, corresponding to SEQ ID No. 2.

The tumour specific promoter elements of the invention, i.e. nucleotide sequences conferring tumour specificity of promoter activity to a promoter, are preferably comprised in a promoter region functionally arranged in 5′ to a transgene, e.g. in an expression cassette containing a transgene. Accordingly, the tumour specific promoter elements of the invention are preferably comprised in a promoter region, or the promoter elements of the invention are promoter regions, which are in functional arrangement to a coding sequence, e.g. a transgene, conferring tumour specific transcription of the transgene to the promoter region, e.g. transcription of the transgene from the expression cassette containing the tumour specific promoter moment functionally linked to the transgene.

The specificity of the promoter elements of the invention to be tumour specific, i.e. to initiate transcription of a nucleotide sequence functionally linked to the promoter comprising or consisting of a tumour specific promoter element has been selected for by a selection method comprising at least three, preferably four, consecutive screening steps. By this method, tumour specific promoter elements could be selected that do not initiate transcription of the transgene in the spleen.

For the purpose of the invention, the term transgene comprises both heterologous and homologous genes, e.g. of the human tumour patient or of the bacterial vector. Preferred transgenes are nucleic acid sequences encoding a cytokine, e.g. selected from interleukins, preferably selected from IL-2, IL-4, IL-12, IL-21, IFN-λ, IFN-α, IFN-β, and IFN-γ, GM-CSF, TNF-α, TGF-β, nucleic acid sequences encoding an angiogenesis inhibitor, e.g. selected from thrombospondin-1, endostatin, and angiopoietin-2, nucleic acid sequences encoding a bacterial toxin, e.g. selected from, Shiga-like toxin, α-toxin and/or Parton-Valentine leukocidin of Staphylococcus aureus, and nucleic acid sequences encoding a prodrug converting enzyme, e.g. nucleic acid sequences encoding carboxypeptidase G2, and/or encoding purine-deoxynucleoside phosphorylase, and/or nucleic acid sequences encoding genes such as colicin, cytolysine and/or cytosine desaminase.

Bacterial vectors are selected among Gram-positive bacteria, preferably among Gram-negative bacteria, most preferably attenuated invasive bacteria. The bacterial vectors of the invention are genetically manipulated to contain an expression cassette containing a coding sequence for a transgene under the control of a bacterial promoter region containing a tumour specific promoter element of the invention. Preferably, the expression cassette is integrated into the bacterial genome. Less preferred, the expression cassette is contained in a plasmid present within the bacterial vector.

The gram-negative bacteria are for example E. coli, Salmonella spp., e.g. Salmonella enterica serovar Typhimurium, like strain SL7207, e.g. Salmonella enterica serovar Typhi, like strain Ty21a, Shigella spp., Yersinia spp., and Vibrio cholerae. Examples for gram-positive bacteria are Bacillus spp., e.g. Bacillus subtilis, Clostridium spp., Listerium monocytogenes, and Mycobacterium spp., e.g. strain BCG. Commensal bacteria are for example E. coli, Lactobacillus spp., Lactococcus spp., and Streptococcus gordonii.

Within the terms of this disclosure, the expression “attenuated invasive bacteria” especially refers to attenuated strains of E. coli, Listerium monocytogenes, Salmonella enterica serovar. typhimurium, Shigella flexneri, Yersinia pseudotuberculosis, and attenuated strains of further invasive bacteria as well as of the non-invasive bacterium Vibrio cholerae. The reason for including Vibrio cholerae for the purposes of this disclosure within the term “invasive bacteria” is that for Vibrio cholerae attenuated strains exist, which have been demonstrated to accumulate within tumor tissue (Nature Biotechnology, Volume 22, No 3, March 2004). With reference to the affinity of Vibrio cholerae to tumours, this is a property shared with at least some invasive bacteria, making them useful within the present invention.

In the context of this disclosure, the classification of bacteria useful as the bacterial vector components of the invention as invasive or non-invasive includes the possibility for some bacteria to colonize organ specific tissue intracellularly and extracellularly. This applies for example to Salmonella, that colonize tumour tissue also in an extracellular state.

Further, invasive bacteria can be used to generate the vector component of the invention which originally are non-invasive bacteria but which have been rendered invasive by genetic manipulation, e.g. by introduction of coding sequences for invasion promoting factors, e.g. the invasin gene (inv) derived from Yersinia pseudotuberculosis, e.g. by expression in E. coli.

Further exemplary bacteria suitable for producing the bacterial vector according to the invention are: Agrobacterium e.g. Agrobacterium tumefaciens; Bacillus e.g. Bacillus cereus, Bacillus subtilis, Bacillus thuringiensis, Bacillus weihenstephanensis; Bartonella e.g. Bartonella henselae, Bartonella schoenbuchensis; Bdellovibrio e.g. Bdellovibrio bacteriovorus, Bdellovibrio starrii, Bdellovibrio stolpii; Bifidobacterium e.g. Bifidobacterium adolescentis, Bifidobacterium bifidum, Bifidobacterium lactis, Bifidobacterium longum; Bordetella e.g. Bordetella pertussis; Borrelia e.g. Borrelia burgdorferi; Brucella e.g. Brucella abortus, Brucella bronchiseptica; Burkholderia e.g. Burkholderia cenocepacia, Burkholderia fungorum, Burkholderia mallei, Burkholderia pseudomallei; Campylobacter e.g. Campylobacter fecalis, Campylobacter pylori, Campylobacter sputorum; Chlamydia e.g. Chlamydia pneumoniae, Chlamydia psittaci, Chlamydia trachomatis; Clostridium e.g. Clostridium difficile, Clostridium novyi, Clostridium oncolyticum, Clostridium perfringens, Clostridium sporogenes, Clostridium tetani; Corynebacterium e.g. Corynebacterium diphtheriae, Corynebacterium glutamicum, Corynebacterium jeikeium; Edwardsiella e.g. Edwardsiella hoshinae, Edwardsiella ictaluri, Edwardsiella tarda; Enterobacter e.g. Enterobacter aerogenes, Enterobacter cloacae, Enterobacter sakazakii; Enterococcus e.g. Enterococcus avium, Enterococcus faecalis, Enterococcus faecium, Enterococcus gallinarum; Escherichia e.g. Escherichia coli; Eubacterium e.g. Eubacterium lentum, Eubacterium nodatum, Eubacterium timidum; Helicobacter e.g. Helicobacter pylori; Klebsiella e.g. Klebsiella oxytoca, Klebsiella pneumoniae; Lactobacillus e.g. Lactobacillus bulgaricus, Lactobacillus casei, Lactobacillus delbrueckii, Lactobacillus plantarum; Lactobacterium e.g. Lactobacterium fermentum; Lactococcus e.g. Lactococcus lactis, Lactococcus plantarum; Legionella e.g. Legionella pneumophila; Listeria e.g. Listeria innocua, Listeria ivanovii, Listeria monocytogenes; Microbacterium e.g. Microbacterium arborescens, Microbacterium lacticum; Mycobacterium e.g. Bacille Calmette-Guerin (BCG), Mycobacterium avium, Mycobacterium bovis, Mycobacterium paratuberculosis, Mycobacterium tuberculosis; Neisseria e.g. Neisseria gonorrhoeae, Neisseria lactamica, Neisseria meningitidis; Pasteurella e.g. Pasteurella haemolytica, Pasteurella multocida; Salmonella e.g. Salmonella bongori, Salmonella enterica ssp.; Shigella e.g. Shigella dysenteriae, Shigella flexneri, Shigella sonnei; Staphylococcus e.g. Staphylococcus aureus, Staphylococcus lactis, Staphylococcus saprophyticus; Streptococcus e.g. Streptococcus gordonii, Streptococcus lactis, Streptococcus pneumoniae, Streptococcus pyogenes, Streptococcus salivarius; Treponema e.g. Treponema denticola, Treponema pallidum; Vibrio e.g. Vibrio cholerae; Yersinia e.g. Yersinia enterocolitica, Yersinia pseudotuberculosis, including S1-strains devoid of Hfr factors and of pili of these bacteria, especially S1-strains of E. coli.

### DETAILED DESCRIPTION OF THE INVENTION

The invention is now described in greater detail with reference to the figures, wherein

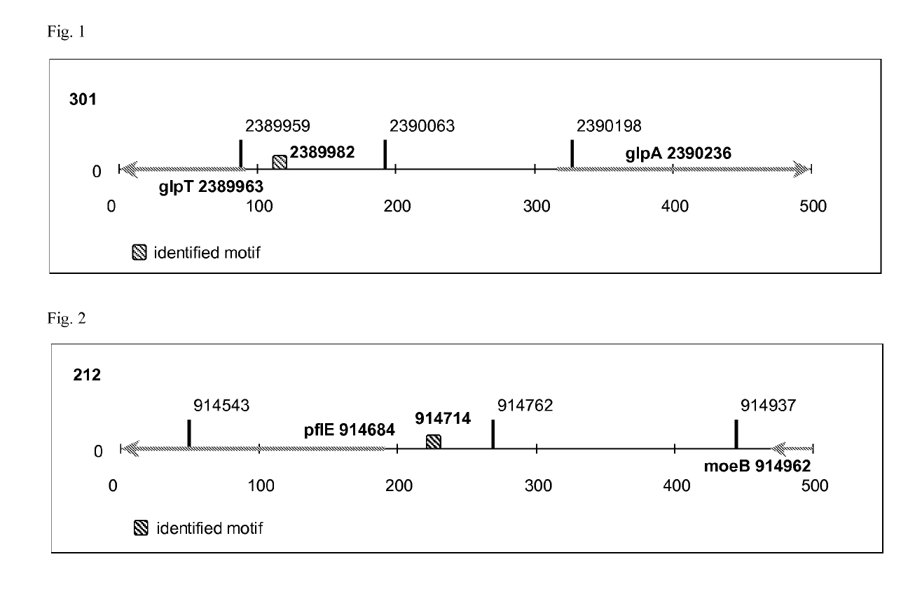

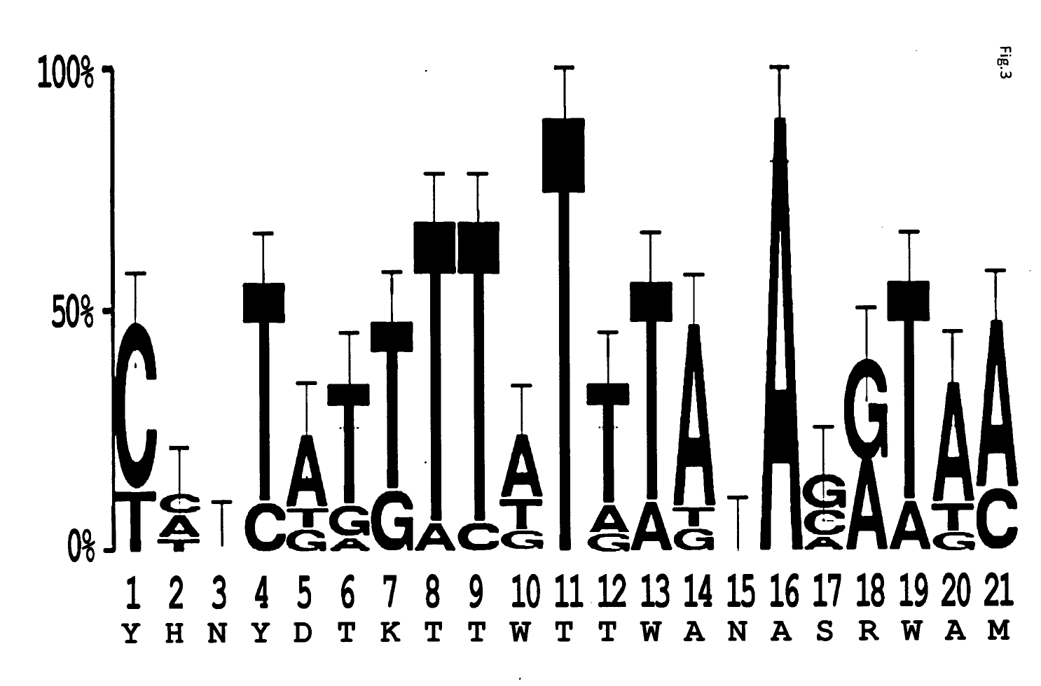

FIGS. 1 and 2 schematically show nucleotides identified by Arrach et al. in relation to the motif of the invention,

FIG. 3 shows a graph indicating the relative prevalence of nucleotides in the motif of the tumour specific promoter element,

FIG. 4 is a graph according to FIG. 3 with the best matching nucleotide sequence, and

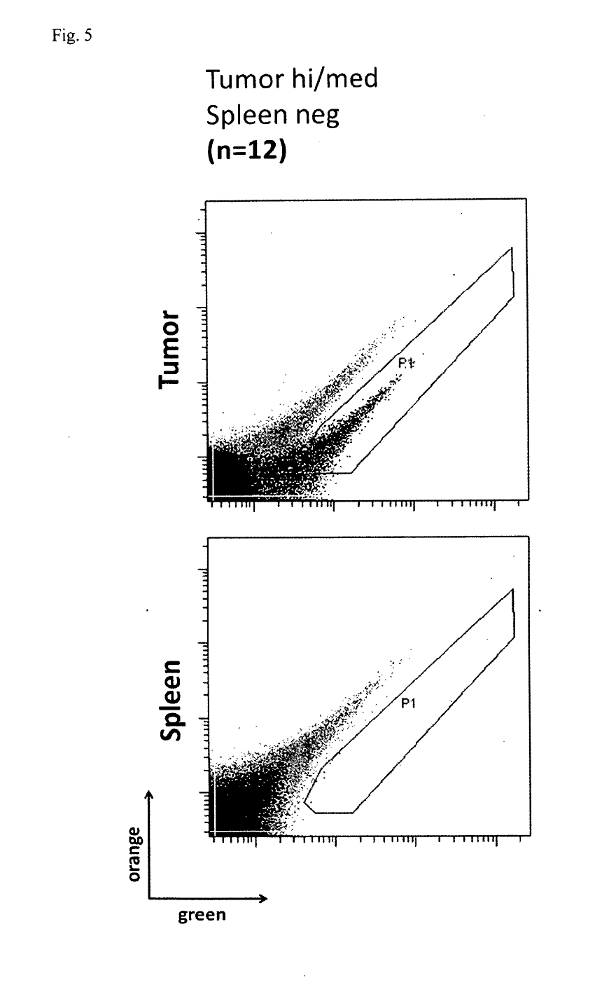

FIG. 5 shows FACS measurement results of reporter gene expression from bacterial vectors of the invention in tumour and spleen, respectively.

In detail, a library of genome fragments obtained from Salmonella typhimurium was generated by arranging genome fragments in 5′ to a reporter gene devoid of a promoter. The reporter gene used was gfp-ova according to Bumann, Mol. Microbiol., 1269-1283 (2002). The reporter gene product is short-lived in order to prevent accumulation of protein in bacterial cells that could occur by induction from a weak promoter, which was not intended to be isolated. According to standard procedures, the expression cassettes containing the reporter gene and containing, in 5′ to the reporter gene, a genomic fragment, was transformed into Salmonella typhimurium SL7207 to generate the bacterial library.

An aliquot of the bacterial library was injected directly, i.e. intra-tumourally, into an established subcutaneous CT26 tumour in a mouse in order to avoid a selection of bacteria by migration into the tumour, that was e.g. observed for systemic application of bacteria.

One day after intra-tumoural infection, the tumour was removed and homogenized. Homogenate was sorted by FACS using two colour flow-cytometry to distinguish reporter gene expressing bacteria from autofluorescent debris.

For this first FACS selection, a very broad region was collected to enrich for GFP-OVA-positive clones, in which presence of the reporter gene product indicates the content of a tumour specific promoter element. Collected bacteria were grown overnight on LB agar plates. Of these selected clones, suspensions were made in LB medium. Aliquots of these bacteria were used for a second intra-tumoural infection as described above, and FACS selection of reporter—positive clones from tumour homogenate. For the second FACS selection, more restrictive settings were used in order to select for clones with high expression. The selected bacteria were plated onto agar plates and grown overnight.

The colonies were scraped off the plates and resuspended and then used for infection of a tumour-free mouse i.v. in order to select for GFP-OVA-negative clones from spleen. One day after infection, the spleen was removed and homogenized. From the homogenate, bacteria that were negative for the reporter gene expression were selected by FACS sorting. In this way, it was ensured that promoter elements that had been selected before for their activity in solid tumour tissue, would not show activity in spleen, which organ was taken as a representative for non-tumour tissue.

Finally, bacteria selected from the FACS sorting as reporter-negative in spleen, were grown on LB-plates and resuspended collectively. An aliquot of the bacterial suspension was used for infection of a tumour bearing mouse i.v. As described above, the tumour was removed on the next day and homogenized. From the homogenate, strong expression of reporter gene was used by restricting the settings of FACS to select for GFP-OVA-high expressing clones. As a result, about 3,000 colonies could be plated from the selected fraction of bacteria.

Inserts from bacterial colonies that were grown of the finally selected bacterial fraction were sequenced. For further selecting bacterial clones for inserts driving transcription of the reporter gene in a solid tumour, but essentially not inducing transcription of the reporter gene in non-tumour tissue, e.g. in spleen, individual bacterial clones were again used to infect a tumour bearing mouse i.v. Recovery of bacterial vectors from tumour homogenate and spleen, respectively, was followed by FACS analysis of GFP-OVA expression. The clones were selected by choosing high reporter gene expression in tumour, in combination with little or no expression of the reporter gene in spleen.

The multi-step selection method for the tumour specific promoter elements of the invention demonstrates that induction of transgene transcription is controlled to be tumour specific in combination with essentially no activation of transgene transcription in a non-tumour environment, e.g. in spleen.

In the individual clones, the following tumour specific promoter elements were identified:

(SEQ ID No. 6)
ttttgtttaaaaaaaatacag,
(SEQ ID No. 8)
ttacctctaaataaaatatgg,
(SEQ ID No. 10)
cactgtgattttttgaggtaa,
(SEQ ID No. 12)
ttacgtgtaaaaaaacaaatg,
(SEQ ID No. 14)
ttacttttaaaaaaccaactg,
(SEQ ID No. 16)
tctcattttttctcaacgtga,
(SEQ ID No. 18)
gcacgttttaataaattgggg,
(SEQ ID No. 20)
cagcattttgtgtataaatta,
(SEQ ID No. 22)
taatctttatatgaaataaga,
(SEQ ID No. 24)
ttgtatgttatttgtacaaac,
(SEQ ID No. 26)
ccatattttatttagaggtaa,
(SEQ ID No. 28)
gtatctttttataaaacacaa.

Preferably, a promoter region contains a tumour specific promoter element in a distance of 10 to 600 nucleotides in 5′ to the ribosome binding site of an expression cassette containing an orf (open reading frame nucleotide sequence) of a transgene.

Preferably, the promoter regions containing the tumour specific promoter elements are

### SEQ ID No. 5 for SEQ ID No. 6, SEQ ID No. 7 for SEQ ID No. 8,

### SEQ ID No. 9 for SEQ ID No. 10, SEQ ID No. 11 for SEQ ID No. 12,

### SEQ ID No. 13 for SEQ ID No. 14, SEQ ID No. 15 for SEQ ID No. 16,

### SEQ ID No. 17 for SEQ ID No. 18, SEQ ID No. 19 for SEQ ID No. 20,

### SEQ ID No. 21 for SEQ ID No. 22, SEQ ID No. 23 for SEQ ID No. 24,

SEQ ID No. 25 for SEQ ID No. 26, and SEQ ID No. 27 for SEQ ID No. 28, respectively.

It was found that the multitude of single nucleotides, which were identified by Arrach et al., do not motivate nor guide the identification of the tumour-specific motif of SEQ ID No. 1. As an example, FIG. 1 shows the motif, which as indicated extends for 21 nt from nt 2389982, in relation to the nucleotides (nt) 2389959, 2390063 and 2390198 between gipT (starting at nt 2389963) and glpA (starting at nt 2390236). It becomes clear that the nucleotides which Arrach et al identified as putative tumour-specific nucleotides are located at a distance from the motif of the invention.

As a further comparison, clone No. 10, which is preferred in Arrach et al as a tumour-specific clone, FIG. 2 shows the relation of the nucleotides identified by Arrach et al to the motif. The nucleotides Nos. 914543, 914762, and 914937, located between pflE (starting at nt 914684) and moeB (extending to nt 914962) are spaced from the motif, which extends from nt 914714 by 21 nt.

Further, no motif could be derived from the multitude of single nucleotides that were identified by Arrach et al. In an attempt to deduce a promoter element from the single nucleotide positions of Arrach et al, regions of 800 nt around (400 nt in both directions) these single nucleotides were selected. This length of 800 nt is larger than the 300 to 500 nt used in the experiments of Arrach et al, and larger than the average length of segments used during the analysis that forms the basis of the present invention. Due to the larger length of segments, the probability of identifying the motif in this approach is even increased. This analysis was done with the same parameters as those used during the preparation of the invention. It was found that no common sequence or motif corresponding to the motif of the invention could be derived.

### Example

### Bacterial Vector for Tumour Specific Expression of a Transgene

As a control experiment for expression of the transgene in tumour only, bacterial Salmonella typhimurium clones harbouring the tumour specific promoter elements of the invention as nucleic acid sequences integrated into the expression cassette containing the reporter gene gfp-ova were used to intravenously infect BALB/c mice bearing CT26 tumours. 24 hours post infection, tumours, spleens and livers were removed, homogenized and prepared for FACS analysis. As used during the selection method, a volume of 250 μL homogenate in PBS was measured by FACS. It could be shown that transgene expression was obtained for the promoter elements of the invention in tumour tissue, whereas essentially no transgene expression was determined for spleen and liver.

In all animal experiments, generally 6- to 8-week-old female BALB/c mice were used. Injections were subcutaneously at the abdomen with 5×10⁵ CT26 cells. For infection with bacterial vectors, mice that had developed tumours of approximately 4-7 mm diameter were injected with about 5×10⁶ CFU of the bacterial vector in suspension in phosphate buffered saline (PBS). For FACS measurements, homogenates were diluted 1:10 for spleen and liver homogenates, and 1:100 for tumours, respectively, in 0.1% v/v Triton-X100/PBS containing 2 mM EDTA and filtered using a 30 μm Celltrix filter. Sorting or analysis by FACS was made on a FACSAria or a LSRII (Becton Dickinson, USA) flow cytometer.

An exemplary result for a clone of S. typhimurium containing the gfp-ova gene in 3′ to a promoter region containing a promoter element of the invention is shown in FIG. 5 for the tumour homogenate and the spleen homogenate, respectively. The fluorescence of orange against green was measured for discriminating autofluorescent cellular debris, which gives a higher orange-to-green fluorescence emission ratio compared to bacterial vectors showing reporter gene expression (gated in P1). The inserted field circumscribes the events that were assigned to high tumour specificity of induction of reporter gene transcription, in combination with essentially no induction of reporter gene transcription in spleen.

This example demonstrates that the promoter elements of the invention, when comprised in a promoter region of an expression cassette, result in the transcription of the orf which is functionally linked to the promoter region at presence of the bacterial vector in tumour tissue, whereas at presence of the bacterial vector in spleen, essentially no transcription is effected.

1. Pharmaceutical composition suitable for use as a medicament against tumours, the composition comprising a bacterial vector containing an expression cassette comprising a transgene encoding nucleotide sequence functionally linked to a bacterial promoter, characterized in that the bacterial promoter comprises a promoter element having a nucleotide sequence corresponding to the sequence of nucleotides of the matrix

| Position | A | C | G | T |
|---|---|---|---|---|
| 1 | 0.00 | 461.86 | 0.00 | 173.20 |
| 2 | 85.48 | 85.48 | 0.00 | 64.11 |
| 3 | 14.14 | 14.14 | 35.36 | 14.14 |
| 4 | 0.00 | 131.60 | 0.00 | 592.18 |
| 5 | 241.75 | 0.00 | 69.07 | 69.07 |
| 6 | 45.21 | 0.00 | 90.42 | 361.68 |
| 7 | 0.00 | 0.00 | 173.20 | 461.86 |
| 8 | 78.03 | 0.00 | 0.00 | 780.25 |
| 9 | 0.00 | 78.03 | 0.00 | 780.25 |
| 10 | 203.35 | 0.00 | 33.89 | 135.56 |
| 11 | 0.00 | 0.00 | 0.00 | 1100.00 |
| 12 | 90.42 | 0.00 | 45.21 | 361.68 |
| 13 | 131.60 | 0.00 | 0.00 | 592.18 |
| 14 | 510.36 | 0.00 | 56.71 | 56.71 |
| 15 | 52.52 | 10.50 | 31.51 | 21.01 |
| 16 | 1100.00 | 0.00 | 0.00 | 0.00 |
| 17 | 50.51 | 101.02 | 126.27 | 0.00 |
| 18 | 251.49 | 0.00 | 301.79 | 0.00 |
| 19 | 131.60 | 0.00 | 0.00 | 592.18 |
| 20 | 361.68 | 0.00 | 45.21 | 90.42 |
| 21 | 461.86 | 173.20 | 0.00 | 0.00 |

with the proviso that the sum of the matrix values for each nucleotide is at least 7460, or its reverse complementary sequence.

2. Pharmaceutical composition according to claim 1, characterized in that the nucleotide sequence of the promoter element further corresponds to YHNYDTKTTWTTWANASRWAM (SEQ ID No. 1), wherein non-standard nucleotides are represented as follows: R is G or A, Y is T or C, M is A or C, K is G or T, W is A or T, S is G or C, B is T or G or C, V is A or G or C, H is A or T or C, D is A or T or G, and N is A or T or G or C.

3. Pharmaceutical composition according to claim 1, wherein the nucleotide sequence of the promoter element corresponds to SEQ ID No. 3.

4. Pharmaceutical composition according to claim 1, wherein the nucleotide sequence of the promoter element corresponds to SEQ ID No. 4.

5. Pharmaceutical composition according to claim 1, wherein the nucleotide sequence of the promoter element is selected from the group consisting of SEQ ID No. 6, SEQ ID No. 8, SEQ ID No. 10, SEQ ID No. 12, SEQ ID No. 14, SEQ ID No. 16, SEQ ID No. 18, SEQ ID No. 20, SEQ ID No. 22, SEQ ID No. 24, SEQ ID No. 26, and SEQ ID No. 28.

6. Pharmaceutical composition according to claim 1, wherein the nucleotide sequence of the promoter element is comprised in a sequence selected from the group consisting of SEQ ID No. 5, SEQ ID No. 7, SEQ ID No. 9, SEQ ID No. 11, SEQ ID No. 13, SEQ ID No. 15, SEQ ID No. 17, SEQ ID No. 19, SEQ ID No. 21, SEQ ID No. 23, SEQ ID No. 25, and SEQ ID No. 27.

7. Pharmaceutical composition according to claim 1, wherein the transgene is selected from the group comprising genes of human or bacterial origin, pro-drug converting enzyme genes, genes encoding interleukins, IL-2, IL-12, IL-21, IFN-λ, IFN-α, IFN-β, and IFN-γ, GM-CSF, TNF-α, and/or TGF-β, nucleic acid sequences encoding an angiogenesis inhibitor, thrombospondin-1, endostatin, and/or angiopoietin-2, nucleic acid sequences encoding a bacterial toxin, colicin, shiga-like toxin, α-toxin and/or Parton-Valentine leukocidin of Staphylococcus aureus, and nucleic acid sequences encoding cytosine deaminase, carboxypeptidase G2, and/or purine-deoxynucleoside phosphorylase.

8. Bacterial vector comprising a transgene encoding nucleotide sequence under the control of a promoter for use as a medicament against solid tumours characterized in that the bacterial promoter comprises a promoter element having a nucleotide sequence corresponding to the sequence of nucleotides of the position weight matrix

| Position | A | C | G | T |
|---|---|---|---|---|
| 1 | 0.00 | 461.86 | 0.00 | 173.20 |
| 2 | 85.48 | 85.48 | 0.00 | 64.11 |
| 3 | 14.14 | 14.14 | 35.36 | 14.14 |
| 4 | 0.00 | 131.60 | 0.00 | 592.18 |
| 5 | 241.75 | 0.00 | 69.07 | 69.07 |
| 6 | 45.21 | 0.00 | 90.42 | 361.68 |
| 7 | 0.00 | 0.00 | 173.20 | 461.86 |
| 8 | 78.03 | 0.00 | 0.00 | 780.25 |
| 9 | 0.00 | 78.03 | 0.00 | 780.25 |
| 10 | 203.35 | 0.00 | 33.89 | 135.56 |
| 11 | 0.00 | 0.00 | 0.00 | 1100.00 |
| 12 | 90.42 | 0.00 | 45.21 | 361.68 |
| 13 | 131.60 | 0.00 | 0.00 | 592.18 |
| 14 | 510.36 | 0.00 | 56.71 | 56.71 |
| 15 | 52.52 | 10.50 | 31.51 | 21.01 |
| 16 | 1100.00 | 0.00 | 0.00 | 0.00 |
| 17 | 50.51 | 101.02 | 126.27 | 0.00 |
| 18 | 251.49 | 0.00 | 301.79 | 0.00 |
| 19 | 131.60 | 0.00 | 0.00 | 592.18 |
| 20 | 361.68 | 0.00 | 45.21 | 90.42 |
| 21 | 461.86 | 173.20 | 0.00 | 0.00 |

with the proviso that the sum of the matrix values for each nucleotide is at least 7460, or its reverse complementary sequence.

9. Bacterial vector according to claim 8, characterized in that the nucleotide sequence of the promoter element further corresponds to YHNYDTKTTWTTWANASRWAM (SEQ ID No. 1), wherein non-standard nucleotides are represented as follows: R is G or A, Y is T or C, M is A or C, K is G or T, W is A or T, S is G or C, B is T or G or C, V is A or G or C, H is A or T or C, D is A or T or G, and N is A or T or G or C.

10. Bacterial vector according to claim 9, characterized in that the nucleotide sequence encoding the transgene is essentially not transcribed when the bacterial vector is present in spleen or liver tissue.

11. Bacterial vector according to claim 8, wherein the nucleotide sequence of the promoter element is selected from the group consisting of SEQ ID No. 3, SEQ ID No. 4, SEQ ID No. 6, SEQ ID No. 8, SEQ ID No. 10, SEQ ID No. 12, SEQ ID No. 14, SEQ ID No. 16, SEQ ID No. 18, SEQ ID No. 20, SEQ ID No. 22, SEQ ID No. 24, SEQ ID No. 26, and SEQ ID No. 28.

12. Bacterial vector according to claim 8, wherein the nucleotide sequence of the promoter element is comprised in a sequence selected from the group consisting of SEQ ID No. 5, SEQ ID No. 7, SEQ ID No. 9, SEQ ID No. 11, SEQ ID No. 13, SEQ ID No. 15, SEQ ID No. 17, SEQ ID No. 19, SEQ ID No. 21, SEQ ID No. 23, SEQ ID No. 25, and SEQ ID No. 27.

13. Bacterial vector according to one claim 8, wherein the transgene is selected from the group comprising homologous genes, pro-drug converting enzyme genes, genes encoding interleukins, IL-2, IL-12, IL-21, IFN-λ, IFN-α, IFN-β, and IFN-γ, GM-CSF, TNF-α, and/or TGF-β, nucleic acid sequences encoding an angiogenesis inhibitor, thrombospondin-1, endostatin, and/or angiopoietin-2, nucleic acid sequences encoding a bacterial toxin, colicin, shiga-like toxin, α-toxin and/or Parton-Valentine leukocidin of Staphylococcus aureus, and nucleic acid sequences encoding a prodrug converting enzyme, cytosine deaminase, carboxypeptidase G2, and/or purine-deoxynucleoside phosphorylase.

14. Method for the production of a pharmaceutical composition comprising a bacterial vector for use as a medicament for the treatment of solid tumours, characterized by genetically manipulating a bacterial vector by introducing a nucleotide construct containing an expression cassette for a transgene, which expression cassette contains a bacterial promoter comprising a promoter element having a nucleotide sequence corresponding to corresponding to the sequence of the position weight matrix

| Position | A | C | G | T |
|---|---|---|---|---|
| 1 | 0.00 | 461.86 | 0.00 | 173.20 |
| 2 | 85.48 | 85.48 | 0.00 | 64.11 |
| 3 | 14.14 | 14.14 | 35.36 | 14.14 |
| 4 | 0.00 | 131.60 | 0.00 | 592.18 |
| 5 | 241.75 | 0.00 | 69.07 | 69.07 |
| 6 | 45.21 | 0.00 | 90.42 | 361.68 |
| 7 | 0.00 | 0.00 | 173.20 | 461.86 |
| 8 | 78.03 | 0.00 | 0.00 | 780.25 |
| 9 | 0.00 | 78.03 | 0.00 | 780.25 |
| 10 | 203.35 | 0.00 | 33.89 | 135.56 |
| 11 | 0.00 | 0.00 | 0.00 | 1100.00 |
| 12 | 90.42 | 0.00 | 45.21 | 361.68 |
| 13 | 131.60 | 0.00 | 0.00 | 592.18 |
| 14 | 510.36 | 0.00 | 56.71 | 56.71 |
| 15 | 52.52 | 10.50 | 31.51 | 21.01 |
| 16 | 1100.00 | 0.00 | 0.00 | 0.00 |
| 17 | 50.51 | 101.02 | 126.27 | 0.00 |
| 18 | 251.49 | 0.00 | 301.79 | 0.00 |
| 19 | 131.60 | 0.00 | 0.00 | 592.18 |
| 20 | 361.68 | 0.00 | 45.21 | 90.42 |
| 21 | 461.86 | 173.20 | 0.00 | 0.00 |

with the proviso that the sum of the matrix values for each nucleotide is at least 7460, or its reverse complementary sequence.

15. Method according to claim 14, characterized in that the nucleotide sequence of the promoter element further corresponds to YHNYDTKTTWTTWANASRWAM (SEQ ID No. 1), wherein non-standard nucleotides are represented as follows: R is G or A, Y is T or C, M is A or C, K is G or T, W is A or T, S is G or C, B is T or G or C, V is A or G or C, H is A or T or C, D is A or T or G, and N is A or T or G or C.

16. Method according to claim 14, wherein the transgene is selected from the group comprising homologous genes, pro-drug converting enzyme genes, genes encoding interleukins, IL-2, IL-12, IL-21, IFN-λ, IFN-α, IFN-β, and IFN-γ, GM-CSF, TNF-α, and/or TGF-β, nucleic acid sequences encoding an angiogenesis inhibitor, thrombospondin-1, endostatin, and/or angiopoietin-2, nucleic acid sequences encoding a bacterial toxin, colicin, shiga-like toxin, α-toxin and/or Parton-Valentine leukocidin of Staphylococcus aureus, and nucleic acid sequences encoding a prodrug converting enzyme, cytosine deaminase, carboxypeptidase G2, and/or purine-deoxynucleoside phosphorylase.
